(19) United States
(12) Patent Application Publication
(10) Pub. No.: US 2026/0108470 A1
(43) Pub. Date: Apr. 23, 2026

(54) PROTEIN OR POLYPEPTIDE-LOADED EXTRACELLULAR VESICLE DELIVERY PLATFORM AND APPLICATION THEREOF

(71) Applicants: THE FIRST AFFILIATED HOSPITAL OF USTC, ANHUI PROVINCIAL HOSPITAL, Hefei (CN); UNIVERSITY OF SCIENCE AND TECHNOLOGY OF CHINA, Hefei (CN)

(72) Inventors: Linzhao CHENG, Hefei (CN); Senquan LIU, Hefei (CN); Ding MA, Hefei (CN)

(21) Appl. No.: 19/428,281

(22) Filed: Dec. 21, 2025

Related U.S. Application Data
(63) Continuation of application No. PCT/CN2024/073376, filed on Jan. 19, 2024.

(30) Foreign Application Priority Data
Jun. 21, 2023 (CN) 202310754272.X

(51) Int. Cl. A61K 9/50; A61K 38/45; A61K 39/00; A61K 39/395; A61P 35/00; C12N 5/071; C12N 9/12; C12N 15/85

(52) U.S. Cl. CPC A61K 9/5068; A61K 38/45; A61K 39/3955; A61P 35/00; C12N 5/0686; C12N 9/1241; C12N 15/85; A61K 2039/505; C12N 2510/00

(57) ABSTRACT

A plasmid combination for preparing an extracellular vesicle loaded with a target protein or polypeptide, including a pCAG-GOI plasmid and a pMD2.G plasmid, where GOI refers to a gene sequence corresponding to the target protein or the polypeptide. The pCAG-GOI plasmid is a pCAG-cyclic GMP-AMP synthase (cGAS) gene plasmid, a pCAG-cyclization recombinase (Cre) gene plasmid and a combination thereof. An extracellular vesicle loaded with cyclic GMP-AMP synthase, cyclization recombinase or a combination thereof is provided, which is prepared by transfecting a cell with the plasmid combination. An application of the extracellular vesicle in the prevention or treatment of a cancer is further provided.

### CROSS-REFERENCE TO RELATED APPLICATIONS

This application is a continuation of International Patent Application No. PCT/CN2024/073376, filed on Jan. 19, 2024, which claims the benefit of priority from Chinese Patent Application No. 202310754272.X, filed on Jun. 21, 2023. The content of the aforementioned application, including any intervening amendments made thereto, is incorporated herein by reference in its entirety.

### REFERENCE TO AN ELECTRONIC SEQUENCE LISTING

The contents of the electronic sequence listing (Name: SequenceListing.xml; Size: 10,864 bytes; and Date of Creation: Dec. 15, 2025) is herein incorporated by reference in its entirety.

### TECHNICAL FIELD

This application relates to drug delivery, and more particularly to a protein or polypeptide-loaded extracellular vesicle delivery platform and an application thereof.

### BACKGROUND

Protein therapy refers to the application of biomolecules such as exogenous proteins or peptides to regulate cell biological functions and treat diseases. Compared to gene therapy/chemotherapy, protein therapy does not cause permanent genetic changes and serious adverse reactions. Due to the unique properties of proteins themselves, such as large size, hydrophilicity, membrane impermeability and easy degradation, clinically approved protein drug targets are limited to ligands in an extracellular environment or disease targets on a cell surface. However, approximately 70% of human proteins are intracellular proteins.

Currently, intracellular protein delivery systems typically rely on a protein encapsulation carrier such as a cationic liposome, a polymer and an inorganic nanomaterial. However, a nanocarrier (the liposome, the polymer, the inorganic nanomaterial, etc.) often require covalent modifications of the proteins. Moreover, the efficiencies of protein loading and lysosome escape are low. How to enable a highly-efficient intracellular protein delivery remains a challenge. Therefore, there is an urgent need to develop a stable delivery system that can efficiently deliver a target protein into a target cell and exhibit a function under biological conditions.

Extracellular vesicles (EVs) are a type of natural nanoscale particles with a bilayer membrane structure, including exosomes (diameter: 30-200 nm) and microvesicles (diameter: less than 1000 nm). As an important carrier for the cell-cell communication, the EVs participate in a wide variety of processes including immune regulation, inflammation, tumor, metabolic disorder and angiogenesis, and play an important role in regulating the physiological and pathological processes. The EVs have attracted much attention as a potential delivery carrier due to their good structural stability, inherent advantages in transporting bioactive substances and excellent biocompatibility. By artificially loading functional proteins, nucleic acids and small molecule drugs, etc., combined with targeted modification strategies, the EVs can be given cell and tissue targeting specificity, improve drug efficiency, reduce dosing frequency and alleviate side effects, showing great application prospects in the fields of immunotherapy and drug delivery. Engineered EVs still face numerous challenges in satisfying the required therapeutic levels of concentration, stability, safety and biocompatibility.

### SUMMARY

The technical problem to be solved by the present disclosure is how to obtain an extracellular vesicle delivery system that can efficiently carry a biologically-active protein or polypeptide into cells from an engineered extracellular vesicle supply platform.

In order to achieve the above object, the following technical solutions are adopted.

This application provides a plasmid combination for preparing an extracellular vesicle loaded with a target protein or a polypeptide, comprising:
a CAG promoter-gene of interest-containing (pCAG-GOI) plasmid; and
a pMD2.G plasmid;
wherein GOI is a gene sequence corresponding to the target protein or polypeptide.

The CAG promoter consists of the cytomegalovirus (CMV) enhancer fused to the chicken beta-actin promoter.

In some embodiments, the target protein comprises an intracellular protein.

In some embodiments, the pCAG-GOI plasmid is selected from the group consisting of a cyclic GMP-AMP synthase (cGAS) gene plasmid, a pCAG-cyclization recombinase (Cre) gene plasmid and a combination thereof.

This application also provides a cell for preparing an extracellular vesicle loaded with a target protein or a polypeptide, where the cell is transfected with the aforementioned plasmid combination; a human embryonic stem cell and a human body at various stages of formation and development (such as a human germ cell, a fertilized egg or an embryo) are excluded from the cell; and the cell comprises a cell capable of producing the extracellular vesicle for loading a target drug.

In some embodiments, the cell is a human embryonic kidney 293F (HEK293F) cell.

This application further provides an extracellular vesicle, where the extracellular vesicle is prepared by transfecting a cell with the aforementioned plasmid combination; and the GOI is cyclic GMP-AMP synthase (cGAS) gene, cyclization recombinase (Cre) gene or a combination thereof.

This application further provides a method for preventing or treating a cancer in a subject in need thereof, comprising:
administering a therapeutically effective amount of the aforementioned extracellular vesicle to the subject.

This application further provides another method for preventing or treating a cancer in a subject in need thereof, comprising:
administering a therapeutically effective amount of the aforementioned extracellular vesicle to the subject in combination with an inhibitor of an immune checkpoint, an antagonist of the immune checkpoint or a combination thereof.

In some embodiments, the immune checkpoint is selected from the group consisting of cytotoxic T lymphocyte-associated antigen-4 (CTLA-4), programmed death receptor-1 (PD-1), programmed death ligand-1 (PD-L1), indoleamine 2,3-dioxygenase-1 (IDO-1), T cell immunoglobulin and mucin domain-containing protein 3 (TIM-3), lymphocyte activation gene-3 (LAG-3), killer-cell immunoglobulin-like receptor (KIR), glucocorticoid-induced tumor necrosis factor receptor-related protein (GITR), V-domain Ig suppressor of T cell activation (VISTA), tumor necrosis factor receptor superfamily member 9 (4-1BB), tumor necrosis factor receptor superfamily 4 (OX40), B7 homolog 3 protein (B7-H3) and tumor necrosis factor receptor superfamily 7 (CD27); and
the inhibitor and the antagonist are each a small molecule compound or an antibody.

In some embodiments, the cancer is colon cancer or a melanoma.

This application further provides a method for loading a target protein or a target polypeptide into an extracellular vesicle, comprising:
transfecting a cell with the aforementioned plasmid combination to obtain a transfected cell; and
culturing the transfected cell to express the extracellular vesicle loaded with the target protein or the target polypeptide.

In some embodiments, the step of culturing the transfected cell to express the extracellular vesicle loaded with the target protein or the target polypeptide comprises:
(S1) culturing the transfected cell for a period of time, and then collecting and storing a culture supernatant; and
(S2) isolating the extracellular vesicle from the culture supernatant.

In some embodiments, the plasmid combination comprises a pCAG-GOI plasmid and a pMD2.G plasmid, wherein GOI is the gene sequence corresponding to the target protein or the polypeptide.

In some embodiments, the culture supernatant is collected on the fourth day after transfection; the culture supernatant is stored at −80° C. for use; and the extracellular vesicle is isolated from the culture supernatant through ultracentrifugation.

In some embodiments, the ultracentrifugation is performed through steps of:
centrifuging the culture supernatant at 300×g for 10 min to collect a first resultant supernatant into a first centrifuge tube;
centrifuging the first resultant supernatant at 2,000×g for 10 min to collect a second resultant supernatant into a second centrifuge tube;
centrifuging the second resultant supernatant at 100,000×g for 70 min to obtain a third resultant supernatant, followed by removal of the third resultant supernatant to obtain a precipitate;
adding an adequate volume of sterile phosphate buffer saline (PBS) to the precipitate, followed by centrifugation at 100,000× g for 70 min to wash the extracellular vesicle; and
resuspending the extracellular vesicle in PBS, followed by storing at −80° C. for use.

In some embodiments, the cell is a HEK293F cell.

In some embodiments, the extracellular vesicle is prepared by any one of the aforementioned methods.

In some embodiments, a sequence of the pMD2.G plasmid is shown as SEQ ID NO: 1; and a sequence of the pCAG plasmid is shown as SEQ ID NO: 2; wherein the pMD2.G is represented as vesicular stomatitis virus glycoprotein (VSV-G).

Compared to the prior art, the present disclosure has the following beneficial effects.

By means of the dual-plasmid co-transfection of cells, the present disclosure enables a highly-efficient loading of the target protein or the polypeptide into the EVs, further promoting the clinical use of the EVs as a carrier to deliver target substances (such as the protein or the polypeptide) to exert a therapeutic effect. By virtue of good structural stability, inherent advantage of transporting biologically-active substances and excellent biocompatibility, the EVs can significantly reduce systemic toxicities and side effects compared to the traditional delivery carriers. In particular, the extracellular vesicle delivery platform provided herein enables the highly-efficient delivery of intracellular proteins in patients.

### BRIEF DESCRIPTION OF THE DRAWINGS

In order to illustrate the technical solutions in the prior art or in the embodiments of the present disclosure more clearly, the accompanying drawings needed in the description of the prior art or the embodiments of the present disclosure will be briefly described below. Obviously, presented in the accompanying drawings are merely some embodiments, instead of all embodiments, of the disclosure, and other drawings can also be obtained by those skilled in the art based on these accompanying drawings without paying creative effort.

FIG. 1 shows Western Blot (WB) analysis results of the expression of cyclization recombinase (Cre) protein;

FIG. 2 shows the efficiency of engineered extracellular vesicles (EVs) in delivering the Cre protein (expressed by percentage of green fluorescent protein-positive (GFP⁺) cells);

FIGS. 3a-c are transmission electron microscopy (TEM) images of individual groups, where 3a: native EVs; 3b: control EVs; and 3c: cGAS EVs;

FIG. 4 shows particle size distribution of the EVs detected by a nanoparticle tracking analyzer;

FIG. 5 shows particle concentration of the EVs detected by a nanoparticle tracking analyzer;

FIG. 6 shows Western Blot (WB) analysis results of the expression of EVs marked protein;

FIG. 7 shows Western Blot (WB) analysis results of the expression of cGAS protein;

FIG. 8 shows the results of interferon (IFN) expression efficiently induced by EVs loaded with cGAS protein;

FIGS. 9a-9d shows expression levels of individual inflammatory factors in immune cells enhanced by cGAS protein-loaded EVs, where 9a: interferon-β (IFN-β); 9b: CXC chemokine ligand-10 (CXCL10); 9c: tumor necrosis factor-α (TNF-α); and 9d: interleukin-6 (IL-6);

FIG. 10 shows the results of tumor volume changes in mice with colon cancer treated with EVs loaded with cGAS protein;

FIG. 11 shows the results of tumor volume changes in mice with melanoma treated with EVs loaded with cGAS protein;

FIG. 12 shows the results of safety evaluation by hematoxylin and eosin (H&E) staining;

FIG. 13 shows the results of tumor volume changes in mice with colon cancer treated with EVs loaded with cGAS protein combined with an inhibitor of an immune checkpoint; and

FIG. 14 shows the survival of mice with colon cancer treated with EVs loaded with cGAS protein combined with the inhibitor of the immune checkpoint.

### DETAILED DESCRIPTION OF EMBODIMENTS

In order to make the objects, technical solutions and advantages of the present disclosure clearer, the present disclosure will be described clearly and completely with reference to the accompanying drawings in the embodiments of the present disclosure. Obviously, described below are merely some embodiments of the present disclosure, not all embodiments. Based on the embodiments in the present disclosure, all other embodiments obtained by those of ordinary skill in the art without paying creative labor shall fall within the scope of the present disclosure.

The present disclosure will be illustrated below in combination with the specific embodiments. The instruments, reagents and materials used herein are in accordance with the ordinary knowledge and conventional means in the art, and all commercially available. The methods used herein, such as a transmission electron microscopy (TEM) imaging and Western Blot (WB), are in accordance with the ordinary knowledge and conventional means in the art, and can be performed with reference to relevant descriptions in textbooks or relevant literature. For the sake of brevity, there will not be elaborated herein. If it is described herein, the method is performed with reference to the descriptions of the present disclosure.

In some embodiments, a sequence of a pMD2.G plasmid is shown as SEQ ID NO: 1, and a sequence of a CAG plasmid is shown as SEQ ID NO: 2. The pMD2.G is represented as vesicular stomatitis virus glycoprotein (VSV-G). The CAG promoter consists of the cytomegalovirus (CMV) enhancer fused to the chicken beta-actin promoter.

The SEQ ID NO: 1 (i.e., the sequence of the pMD2.G plasmid) is shown as follows:

ATGAAGTGCCTTTTGTACTTAGCCTTTTTATTCATTGGGGTGAATTGCA
AGTTCACCATAGTTTTTCCACACAACCAAAAAGGAAACTGGAAAAATGT
TCCTTCTAATTACCATTATTGCCCGTCAAGCTCAGATTTAAATTGGCAT
AATGACTTAATAGGCACAGCCTTACAAGTCAAAATGCCCAAGAGTCACA
AGGCTATTCAAGCAGACGGTTGGATGTGTCATGCTTCCAAATGGGTCAC
TACTTGTGATTTCCGCTGGTATGGACCGAAGTATATAACACATTCCATC
CGATCCTTCACTCCATCTGTAGAACAATGCAAGGAAAGCATTGAACAAA
CGAAACAAGGAACTTGGCTGAATCCAGGCTTCCCTCCTCAAAGTTGTGG
ATATGCAACTGTGACGGATGCCGAAGCAGTGATTGTCCAGGTGACTCCT
CACCATGTGCTGGTTGATGAATACACAGGAGAATGGGTTGATTCACAGT
TCATCAACGGAAAATGCAGCAATTACATATGCCCCACTGTCCATAACTC
TACAACCTGGCATTCTGACTATAAGGTCAAAGGGCTATGTGATTCTAAC
CTCATTTCCATGGACATCACCTTCTTCTCAGAGGACGGAGAGCTATCAT
CCCTGGGAAAGGAGGGCACAGGGTTCAGAAGTAACTACTTTGCTTATGA
AACTGGAGGCAAGGCCTGCAAAATGCAATACTGCAAGCATTGGGGAGTC
AGACTCCCATCAGGTGTCTGGTTCGAGATGGCTGATAAGGATCTCTTTG
CTGCAGCCAGATTCCCTGAATGCCCAGAAGGGTCAAGTATCTCTGCTCC
ATCTCAGACCTCAGTGGATGTAAGTCTAATTCAGGACGTTGAGAGGATC
TTGGATTATTCCCTCTGCCAAGAAACCTGGAGCAAAATCAGAGCGGGTC
TTCCAATCTCTCCAGTGGATCTCAGCTATCTTGCTCCTAAAAACCCAGG
AACCGGTCCTGCTTTCACCATAATCAATGGTACCCTAAAATACTTTGAG
ACCAGATACATCAGAGTCGATATTGCTGCTCCAATCCTCTCAAGAATGG
TCGGAATGATCAGTGGAACTACCACAGAAAGGGAACTGTGGGATGACTG
GGCACCATATGAAGACGTGGAAATTGGACCCAATGGAGTTCTGAGGACC
AGTTCAGGATATAAGTTTCCTTTATACATGATTGGACATGGTATGTTGG
ACTCCGATCTTCATCTTAGCTCAAAGGCTCAGGTGTTCGAACATCCTCA
CATTCAAGACGCTGCTTCGCAACTTCCTGATGATGAGAGTTTATTTTTT
GGTGATACTGGGCTATCCAAAAATCCAATCGAGCTTGTAGAAGGTTGGT
TCAGTAGTTGGAAAAGCTCTATTGCCTCTTTTTTCTTTATCATAGGGTT
AATCATTGGACTATTCTTGGTTCTCCGAGTTGGTATCCATCTTTGCATT
AAATTAAAGCACACCAAGAAAAGACAGATTTATACAGACATAGAGATGA
ACCGACTTGGAAAGTAA.

The SEQ ID NO: 2 (i.e., the sequence of the CAG plasmid) is shown as follows:

GACATTGATTATTGACTAGTTATTAATAGTAATCAATTACGGGGTCATT
AGTTCATAGCCCATATATGGAGTTCCGCGTTACATAACTTACGGTAAAT
GGCCCGCCTGGCTGACCGCCCAACGACCCCCGCCCATTGACGTCAATAA
TGACGTATGTTCCCATAGTAACGCCAATAGGGACTTTCCATTGACGTCA
ATGGGTGGACTATTTACGGTAAACTGCCCACTTGGCAGTACATCAAGTG
TATCATATGCCAAGTACGCCCCCTATTGACGTCAATGACGGTAAATGGC
CCGCCTGGCATTATGCCCAGTACATGACCTTATGGGACTTTCCTACTTG
GCAGTACATCTACGTATTAGTCATCGCTATTACCATGGGTCGAGGTGAG
CCCCACGTTCTGCTTCACTCTCCCCATCTCCCCCCCCTCCCCACCCCCA
ATTTTGTATTTATTTATTTTTTAATTATTTTGTGCAGCGATGGGGGCGG
GGGGGGGGGGGGCGCGCGCCAGGCGGGGCGGGGCGGGGCGAGGGGCGGG
GCGGGGCGAGGCGGAGAGGTGCGGCGGCAGCCAATCAGAGCGGCGCGCT
CCGAAAGTTTCCTTTTATGGCGAGGCGGCGGCGGCGGCGGCCCTATAAA
AAGCGAAGCGCGCGGCGGGCGGGAGTCGCTGCGTTGCCTTCGCCCCGTG
CCCCGCTCCGCGCCGCCTCGCGCCGCCCGCCCCGGCTCTGACTGACCGC
GTTACTCCCACAGGTGAGCGGGCGGGACGGCCCTTCTCCTCCGGGCTGT
AATTAGCGCTTGGTTTAATGACGGCTCGTTTCTTTTCTGTGGCTGCGTG
AAAGCCTTAAAGGGCTCCGGGAGGGCCCTTTGTGCGGGGGGGAGCGGCT
CGGGGGGTGCGTGCGTGTGTGTGTGCGTGGGGAGCGCCGCGTGCGGCCC
GCGCTGCCCGGCGGCTGTGAGCGCTGCGGGCGCGGCGCGGGGCTTTGTG
CGCTCCGCGTGTGCGCGAGGGGAGCGCGGCCGGGGGCGGTGCCCCGCGG
TGCGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCCCC
CCTGCACCCCCCTCCCCGAGTTGCTGAGCACGGCCCGGCTTCGGGTGCG
GGGCTCCGTGCGGGGCGTGGCGCGGGGCTCGCCGTGCCGGGCGGGGGGT
GGCGGCAGGTGGGGGTGCCGGGCGGGGGGGGGCCGCCTCGGGCCGGGGA
GGGCTCGGGGGAGGGGCGCGGCGGCCCCGGAGCGCCGGCGGCTGTCGAG
GCGCGGCGAGCCGCAGCCATTGCCTTTTATGGTAATCGTGCGAGAGGGC
GCAGGGACTTCCTTTGTCCCAAATCTGGCGGAGCCGAAATCTGGGAGGC
GCCGCCGCACCCCCTCTAGCGGGCGCGGGCGAAGCGGTGCGGCGCCGGC
AGGAAGGAAATGGGCGGGGAGGGCCTTCGTGCGTCGCCGCGCCGCCGTC
CCCTTCTCCATCTCCAGCCTCGGGGCTGCCGCAGGGGGACGGCTGCCTT
CGGGGGGGACGGGGCAGGGCGGGGTTCGGCTTCTGGCGTGTGACCGGCG
GCTCTAGAGCCTCTGCTAACCATGTTCATGCCTTCTTCTTTTTCCTACA
G

The SEQ ID NO: 3 (i.e., the sequence of a CMV plasmid) is shown as follows:

CGTTACATAACTTACGGTAAATGGCCCGCCTGGCTGACCGCCCAACGA
CCCCCGCCCATTGACGTCAATAATGACGTATGTTCCCATAGTAACGCCA
ATAGGGACTTTCCATTGACGTCAATGGGTGGAGTATTTACGGTAAACTG
CCCACTTGGCAGTACATCAAGTGTATCATATGCCAAGTACGCCCCCTAT
TGACGTCAATGACGGTAAATGGCCCGCCTGGCATTATGCCCAGTACATG
ACCTTATGGGACTTTCCTACTTGGCAGTACATCTACGTATTAGTCATCG
CTATTACCATGGTGATGCGGTTTTGGCAGTACATCAATGGGCGTGGATA
GCGGTTTGACTCACGGGGATTTCCAAGTCTCCACCCCATTGACGTCAAT
GGGAGTTTGTTTTGGCACCAAAATCAACGGGACTTTCCAAAATGTCGTA
ACAACTCCGCCCCATTGACGCAAATGGGCGGTAGGCGTGTACGGTGGGA
GGTCTATATAAGCAGAGCT.

The SEQ ID NO: 4 (i.e., the sequence of an elongation factor 1 alpha EF1α plasmid) is shown as follows:

CGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGA
GAAGGTGGCGCGGGGTAAACTGGGAAAGTGATGTCGTGTACTGGCTCCG
CCTTTTTCCCGAGGGTGGGGGAGAACCGTATATAAGTGCAGTAGTCGCC
GTGAACGTTCTTTTTCGCAACGGGTTTGCCGCCAGAACACAGGTAAGTG
CCGTGTGTGGTTCCCGCGGGCCTGGCCTCTTTACGGGTTATGGCCCTTG
CGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAG
GAGCCCCTTCGCCTCGTGCTTGAGTTGAGGCCTGGCCTGGGCGCTGGGG
CCGCCGCGTGCGAATCTGGTGGCACCTTCGCGCCTGTCTCGCTGCTTTC
GATAAGTCTCTAGCCATTTAAAATTTTTGATGACCTGCTGCGACGCTTT
TTTTCTGGCAAGATAGTCTTGTAAATGCGGGCCAAGATCTGCACACTGG
TATTTCGGTTTTTGGGGCCGCGGGCGGCGACGGGGCCCGTGCGTCCCAG
CGCACATGTTCGGCGAGGCGGGGCCTGCGAGCGCGGCCACCGAGAATCG
GACGGGGGTAGTCTCAAGCTGGCCGGCCTGCTCTGGTGCCTGGCCTCGC
GCCGCCGTGTATCGCCCCGCCCTGGGCGGCAAGGCTGGCCCGGTCGGCA
CCAGTTGCGTGAGCGGAAAGATGGCCGCTTCCCGGCCCTGCTGCAGGGA
GCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGAC
TCCACGGAGTACCGGGCGCCGTCCAGGCACCTCGATTAGTTCTCGACCT
TTTGGAGTACGTCGTCTTTAGGTTGGGGGGAGGGGTTTTATGCGATGGA
GTTTCCCCACACTGAGTGGGTGGAGACTGAAGTTAGGCCAGCTTGGCAC
TTGATGTAATTCTCCTTGGAATTTGCCCTTTTTGAGTTTGGATCTTGGT
TCATTCTCAAGCCTCAGACAGTGGTTCAAAGTTTTTTTCTTCCATTTCA
GGTGTCGTGA.

The SEQ ID NO: 5 (i.e., the sequence of a phosphoglycerate kinase (PGK) plasmid) is shown as follows:

GGGTAGGGGAGGCGCTTTTCCCAAGGCAGTCTGGAGCATGCGCTTTA
GCAGCCCCGCTGGGCACTTGGCGCTACACAAGTGGCCTCTGGCCTCGCA
CACATTCCACATCCACCGGTAGGCGCCAACCGGCTCCGTTCTTTGGTGG
CCCCTTCGCGCCACCTTCTACTCCTCCCCTAGTCAGGAAGTTCCCCCCC
GCCCCGCAGCTCGCGTCGTGCAGGACGTGACAAATGGAAGTAGCACGTC
TCACTAGTCTCGTGCAGATGGACAGCACCGCTGAGCAATGGAAGCGGGT
AGGCCTTTGGGGCAGCGGCCAATAGCAGCTTTGCTCCTTCGCTTTCTGG
GCTCAGAGGCTGGGAAGGGGTGGGTCCGGGGGCGGGCTCAGGGGGGGGC
TCAGGGGCGGGGCGGGCGCCCGAAGGTCCTCCGGAGGCCCGGCATTCTG
CACGCTTCAAAAGCGCACGTCTGCCGCGCTGTTCTCCTCTTCCTCATCT
CCGGGCCTTTCG.

### Example 1 Extraction and Identification of Extracellular Vesicles (EVs) Loaded with a Cyclization Recombinase (Cre) Protein

### (1) Cell Transfection and Collection of Culture Supernatants

Human embryonic kidney 293F (HEK293F) cells were cultured in a Union 293 culture medium supplemented with 1× penicillin-streptomycin solution. An appropriate number of HEK293F cells were cultured in a shaking flask until a cell density reached 3 million/mL, at which point they were ready for transfection experiments. By means of using polyethyleneimine (PEI) transfection reagent, the HEK293F cells were transfected with different plasmids according to the manufacturer's instructions, including CAG-Cre+VSV-G (i.e., co-transfection of the pCAG-Cre and pMD2.G plasmids), CMV-Cre+VSV-G (i.e., co-transfection of the pCMV-Cre and pMD2.G plasmids), EF1α-Cre+VSV-G (i.e., co-transfection of the pEF1α-Cre and pMD2.G plasmids) and PGK-Cre+VSV-G (i.e., co-transfection of the pPGK-Cre and pMD2.G plasmids). Their culture supernatants were collected on the fourth day after transfection and stored at −80° C.

### (2) Isolation of the EVs

EVs were isolated from the culture supernatant through an ultracentrifugation. The ultracentrifugation was performed though the following steps. The culture supernatant was centrifuged at 300×g for 10 min, followed by collection of a first resultant supernatant to a first centrifuge tube. The first resultant supernatant was centrifuged at 2,000×g for 10 min, followed by collection of a second resultant supernatant to a second centrifuge tube. Subsequently, the second resultant supernatant was centrifuged at 100,000×g for 70 min to obtain a third resultant supernatant, followed by removal of the third resultant supernatant to obtain a precipitate. An adequate volume of sterile phosphate buffer saline (PBS) was added to the precipitate, followed by centrifugation at 100,000×g for 70 min to wash the EVs. Finally, the EVs were resuspended in PBS and stored at −80° C. for use.

### (3) Identification of the EVs

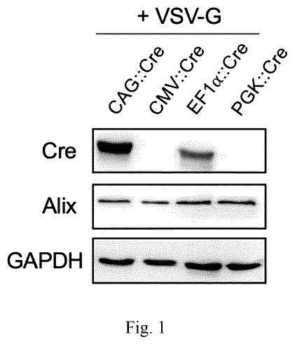

The expression of the Cre protein carried by the EVs from different experimental groups was detected by WB. As shown in FIG. 1, the results indicated that EVs produced by a dual-plasmid co-transfection of a plasmid having a different promoter and the pMD2.G plasmid were capable of delivering the target protein. Among the experimental groups, the dual-plasmid co-transfection of pCAG-GOI and pMD2.G plasmids exhibited an optimal performance. Specifically, EVs prepared by the dual-plasmid co-transfection of the pCAG-Cre and pMD2.G plasmids could efficiently deliver the Cre protein.

### Example 2 Application of the EVs Loaded with the Cre Protein

### (1) Cell Transfection and Collection of Culture Supernatants

The procedure was primarily the same as described in Example 1. The additional steps compared to Example 1 were described below. By means of using PEI transfection reagent, the HEK293F cells were transfected with different plasmids according to the manufacturer's instructions, including VSVG (transfection of the pMD2.G plasmid), CAG-Cre (transfection of the pCAG-Cre plasmid), CMV-Cre (transfection of the pCMV-Cre plasmid), EF1α-Cre (transfection of the pEF1α-Cre plasmid), and PGK-Cre (transfection of the pPGK-Cre plasmid). Their culture supernatants were collected on the fourth day after transfection and stored at −80° C.

### (2) Isolation of the EVs

The procedure was the same as described in Example 1.

### (3) Application of the EVs

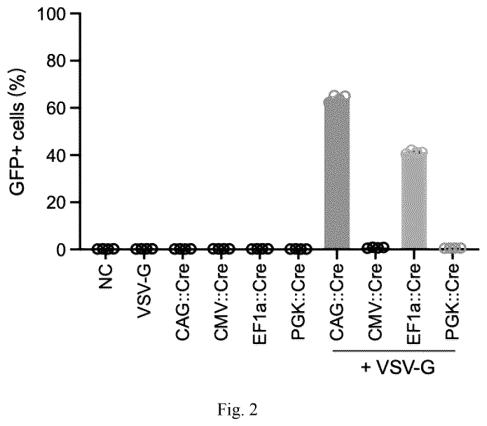

The efficiency of EVs produced by different transfection systems in delivering the Cre protein was evaluated by using a Cre-locus of X (cross)-over in P1 (LoxP) reporter gene assay. The HEK293F cells were transfected with different target plasmid combinations. The EVs were collected and purified to obtain purified EVs. The purified EVs were then added to a reporter cell line having a “LoxP-Stop-LoxP-green fluorescent protein (GFP)” structure to obtain engineered EVs. The delivery efficiency of the engineered EVs, represented by the percentage of green fluorescent protein-positive (GFP) cells, was analyzed through a flow cytometry (If the EVs successfully delivered the Cre protein into the reporter cell line, the LoxP site would be cleaved, causing the reporter cell line to express GFP fluorescence.). NC group was a negative control without EVs addition. As shown in FIG. 2, the results indicated that EVs prepared by co-transfection of the pCAG-Cre and pMD2.G plasmids exhibited the highest delivery efficiency.

### Example 3 Extraction and Identification of the EVs Loaded with a Cyclic GMP-AMP Synthase (cGAS) Protein

### (1) Cell Transfection and Collection of Culture Supernatants

The HEK293F cells were cultured in Union 293 culture medium supplemented with a penicillin-streptomycin solution. An appropriate number of HEK293F cells were seeded and cultured in a shaker until a cell density reached 3×10⁶ cells/mL, at which point they were ready for transfection experiments. By means of using PEI transfection reagent, the HEK293F cells was transfected with different plasmids according to the manufacturer's instructions, including native EVs (no transfection), control (Ctrl) EVs (transfection of pCAG-cGAS plasmid) and cGAS EVs (co-transfection of the pCAG-cGAS and pMD2.G plasmids). Their culture supernatants were collected on the fourth day after transfection and stored at −80° C. for use.

### (2) Isolation of the EVs

EVs were isolated from the culture supernatant through an ultracentrifugation. The ultracentrifugation was performed through the following steps. The culture supernatant was centrifuged at 300×g for 10 min, followed by collection of a first resultant supernatant to a first centrifuge tube. The first resultant supernatant was then centrifuged at 2,000×g for 10 min, followed by connection of a second resultant supernatant to a second centrifuge tube. Subsequently, the second resultant supernatant was centrifuged at 100,000×g for 70 min to obtain a third resultant supernatant, followed by removal of the third resultant supernatant to obtain a precipitate. An adequate volume of sterile PBS was added to the precipitate, followed by centrifugation at 100,000×g for 70 min to wash the EVs. Finally, the EVs were resuspended in PBS and stored at −80° C. for use.

### (3) Identification of the EVs

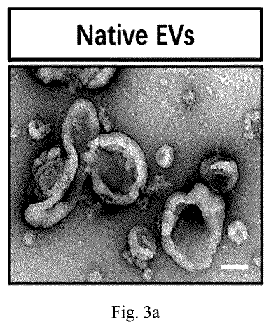

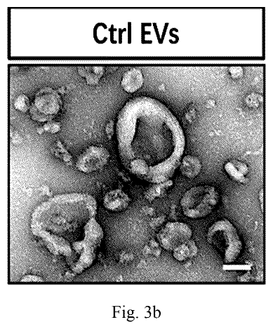

The EVs were examined by using transmission electron microscopy (TEM). As shown in FIGS. 3a-3c, the EVs exhibited a biconcave disc-like morphology with a diameter of ≤200 nm, which was consistent with previous literature reports.

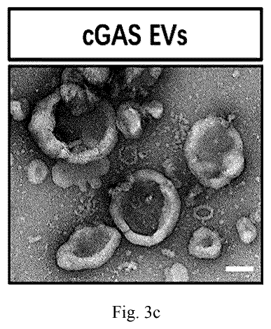

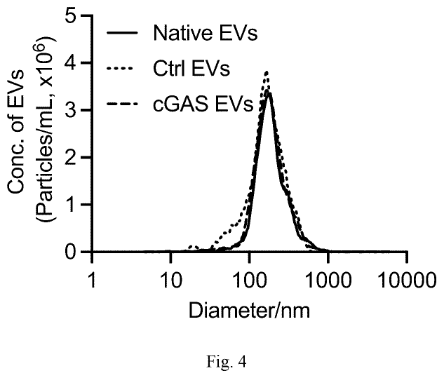

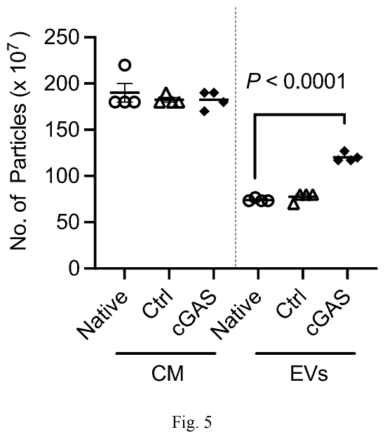

The particle size distribution and concentration of the EVs were detected by nanoparticle tracking analyzer (NTA). As shown in FIGS. 4 and 5, the EVs had a size distribution ranging from 50 nm to 200 nm, which was consistent with previous literature reports.

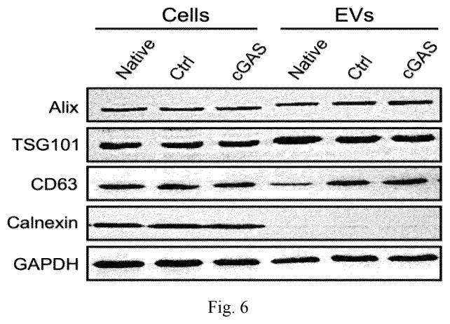

The expression of EVs marked proteins (including apoptosis-linked gene 2-interacting protein X (Alix), tumor susceptibility gene 101 (TSG101) and cluster of differentiation 63 (CD63)) and EVs unmarked protein (i.e., Calnexin) was detected by WB. As shown in FIG. 6, the EVs expressed Alix, TSG101 and CD63, but did not express Calnexin. These results were consistent with previous literature reports.

The expression of the cGAS protein in the EVs was also detected by WB. As shown in FIG. 7, the cGAS protein was detected only in the EVs prepared by the dual-plasmid co-transfection group.

### Example 4 In Vitro Functional Identification of the EVs Loaded with the cGAS Protein

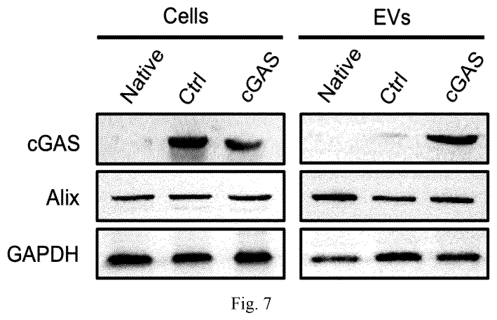

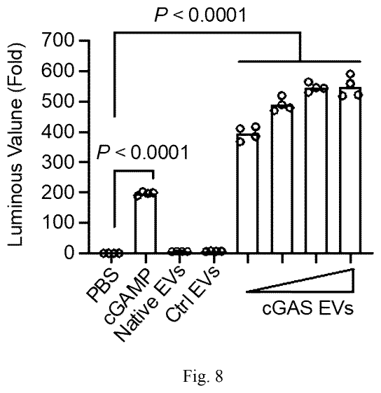

Tohoku hospital pediatrics-1 (THP1)-Lucia™ interferon-stimulated gene (ISG) cells, a commonly used reporter cell line for monitoring interferon (IFN) expression, produced detectable luminescence upon activation of the cGAS-stimulator of interferon gene (STING)-IFN signaling pathway. THP-1 cells were seeded in a 96-well plate, followed by additions of cGAMP (positive control), 10 μL of native EVs, 10 μL of Ctrl EVs, and cGAS EVs, respectively, where the cGAS EVs were at volumes of 1, 2, 5 and 10 μL. After 4 hours of incubation, luciferin culture medium was added to each well, so as to detect an optical density (OD). As shown in FIG. 8, the results demonstrated that the cGAS EVs activated the cGAS-STING-IFN signaling pathway and significantly increased IFN expression in a dose-dependent manner.

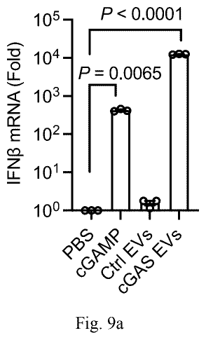

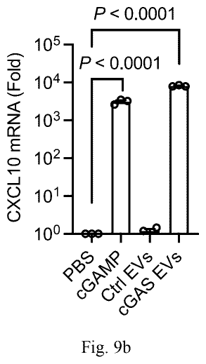

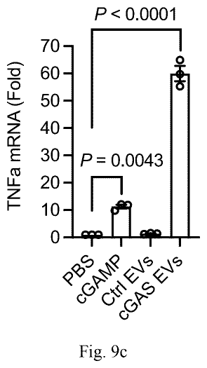

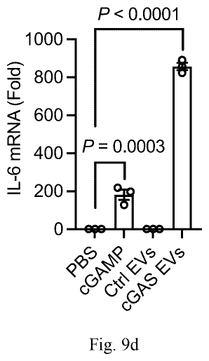

Human monocytic THP-1 cells were seeded in a 24-well plate, followed by additions of cGAMP (positive control), 10 μL of Ctrl EVs and 10 μL of cGAS EVs, respectively. After 4 hours of incubation, total ribonucleic acid (RNA) was collected from each group and subjected to quantitative reverse transcription polymerase chain reaction (qRT-PCR) analysis. As shown in FIGS. 9a-9d, the results indicated that cGAS EVs significantly upregulated the gene expression levels of individual inflammatory factor.

### Example 5 Treatment of Colon Cancer with the EVs Loaded with the cGAS Protein

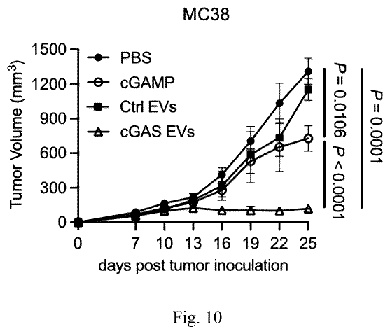

The cGAS EVs were prepared as described in the aforementioned examples. Colon cancer MC38 cells were injected into the right lower flank of female C57 mice to establish a subcutaneous tumor model. When the tumor volume reached approximately 50 mm³, the mice were randomly divided into four groups. The experimental group received intratumoral injections of the cGAS EVs, while the control groups received intratumoral injections of the Ctrl EVs, cGAMP and PBS, respectively. Treatments were performed once every three days for a total of three doses. The tumor volume was measured every three days. On day 25 after the treatment, subcutaneous tumors from each group were harvested and their volumes were recorded. As shown in FIG. 10, the results demonstrated that compared with all control groups, the cGAS EVs significantly inhibited the growth of the colon cancer tumor.

### Example 6 Treatment of Melanoma with the EVs Loaded with the cGAS Protein

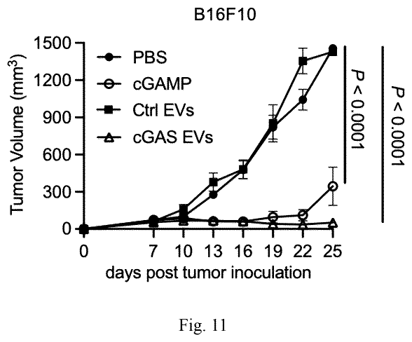

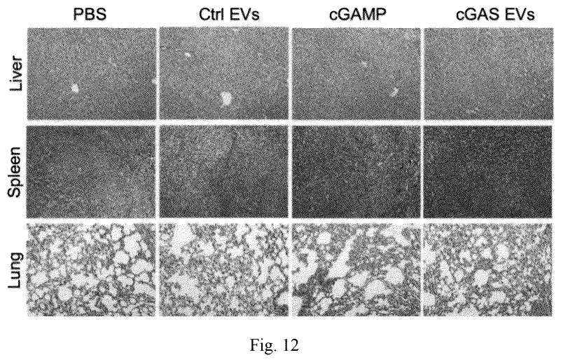

The cGAS EVs were prepared as described in the aforementioned examples. Melanoma B16/F10 cells were injected into the right lower flank of female C57 mice to establish a subcutaneous tumor model. When the tumor volume reached approximately 50 mm³, the mice were randomly divided into four groups. The experimental group received intratumoral injections of the cGAS EVs, while the control groups received intratumoral injections of the Ctrl EVs, cGAMP and PBS, respectively. Treatments were performed once every three days for a total of three doses. Tumor volume was measured every three days. On day 25 post treatment, subcutaneous tumors from each group were harvested and their volumes were recorded. As shown in FIGS. 11 and 12, the results demonstrated that, compared with all control groups, cGAS EVs significantly inhibited melanoma growth without inducing toxic side effects.

### Example 7 Efficacy Enhancement of an Inhibitor of an Immune Checkpoint by the EVs Loaded with the cGAS Protein

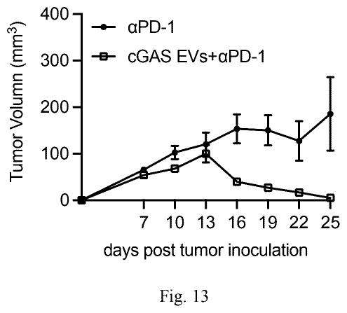

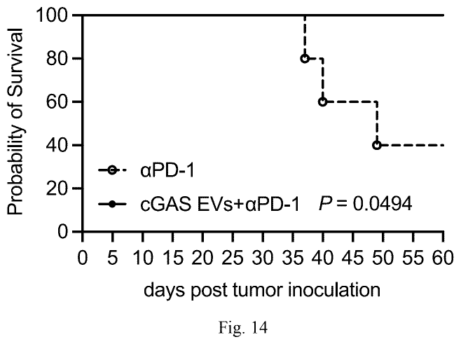

The cGAS EVs were prepared as described in the aforementioned examples. Colon cancer MC38 cells were injected into the right lower flank of female C57 mice to establish a subcutaneous tumor model. When the tumor volume reached approximately 50 mm³, the mice were randomly divided into four groups. The experimental group received intratumoral injection of 50 μL the cGAS EVs and intraperitoneal injection of 200 μg programmed death receptor-1 (PD-1) antibody. The control groups received intratumoral injection of 50 μL the cGAS EVs, 50 μL PBS and intraperitoneal injection of 200 μg PD-1 antibody, respectively. Both intratumoral and intraperitoneal treatments were performed simultaneously once every three days for a total of three doses. Tumor volume was measured every three days. On day 25 post-treatment, subcutaneous tumors from each group were harvested for volume measurement, and mouse survival was monitored. As shown in FIGS. 13 and 14, the results demonstrated that, compared with any single-agent treatment, the cGAS EVs synergistically enhanced the antitumor effect of PD-1 antibody and significantly prolonged mouse survival.

The technical solutions provided herein have the following beneficial effects.

(1) The present disclosure enables a highly-efficient loading of the target protein into the EVs.

(2) The EVs process a biological activity to effectively exert an anti-tumor effect, and have no systemic toxicities or side effects.

(3) The EVs can be combined with the inhibitor of the immune checkpoint to exert a synergistic effect, further promoting the anti-tumor effect and providing patients with more and more effective medication options.

It should be noted that the expressions involving “first”, “second”, etc. in the present disclosure are only for descriptive purposes to differ one entity or operation from another entity or operation, without necessarily requiring or implying any such an actual relationship or sequence between these entities or operations. Furthermore, the terms “include”, “comprise” and any variations thereof are intended to cover a non-exclusive inclusion, such that a process, method, article, or apparatus including a series of elements not only includes those elements but also includes other elements not explicitly listed, or elements inherent to such a process, a method, an article, or an apparatus.

The embodiments disclosed above are merely illustrative of the disclosure, and are not intended to limit the present disclosure. Although the disclosure has been described in detail above with reference to the embodiments, it should be understood by those of ordinary skill in the art, any modifications can still be made to the technical solutions described in the embodiments, or equivalent replacements can be made to the technical features therein. The modifications and equivalent replacements made without departing from the spirit of the disclosure shall fall within the scope of the present disclosure defined by the appended claims.

What is claimed is:

1. A plasmid combination for preparing an extracellular vesicle loaded with a target protein or a target polypeptide, comprising:
a CAG promoter-gene of interest-containing (pCAG-GOI) plasmid; and
a pMD2.G plasmid;
wherein GOI is a gene sequence corresponding to the target protein or the target polypeptide.

2. The plasmid combination of claim 1, wherein the target protein is an intracellular protein.

3. The plasmid combination of claim 1, wherein the pCAG-GOI plasmid is selected from the group consisting of a pCAG-cyclic GMP-AMP synthase (cGAS) gene plasmid, a pCAG-cyclization recombinase (Cre) gene plasmid.

4. A cell for preparing an extracellular vesicle loaded with a target protein or a polypeptide, wherein the cell is transfected with the plasmid combination of claim 1.

5. The cell of claim 4, wherein the cell is a human embryonic kidney 293F (HEK293F) cell.

6. An extracellular vesicle, wherein the extracellular vesicle is prepared by transfecting a cell with the plasmid combination of claim 1; and the GOI is cyclic GMP-AMP synthase (cGAS) gene or cyclization recombinase (Cre) gene.

7. A method for preventing or treating a cancer in a subject in need thereof, comprising:
administering a therapeutically effective amount of the extracellular vesicle of claim 6 to the subject.

8. A method for preventing or treating a cancer in a subject in need thereof, comprising:
administering a therapeutically effective amount of the extracellular vesicle of claim 6 to the subject in combination with an inhibitor of an immune checkpoint, an antagonist of the immune checkpoint or a combination thereof.

9. The method of claim 8, wherein the immune checkpoint is selected from the group consisting of cytotoxic T lymphocyte-associated antigen-4 (CTLA-4), programmed death receptor-1 (PD-1), programmed death ligand-1 (PD-L1), indoleamine 2,3-dioxygenase-1 (IDO-1), T cell immunoglobulin and mucin domain-containing protein 3 (TIM-3), lymphocyte activation gene-3 (LAG-3), killer-cell immunoglobulin-like receptor (KIR), glucocorticoid-induced tumor necrosis factor receptor-related protein (GITR), V-domain Ig suppressor of T cell activation (VISTA), tumor necrosis factor receptor superfamily member 9 (4-1BB), tumor necrosis factor receptor superfamily 4 (OX40), B7 homolog 3 protein (B7-H3) and tumor necrosis factor receptor superfamily 7 (CD27); and
the inhibitor and the antagonist are each a small molecule compound or an antibody.

10. The method of claim 8, wherein the cancer is colon cancer or melanoma.

11. A method for loading a target protein or a target polypeptide into an extracellular vesicle, comprising:
transfecting a cell with the plasmid combination of claim 1 to obtain a transfected cell; and
culturing the transfected cell to express the extracellular vesicle loaded with the target protein or the target polypeptide.

12. The method of claim 11, wherein the step of culturing the transfected cell to express the extracellular vesicle loaded with the target protein or the target polypeptide comprises:
(S1) culturing the transfected cell for a period of time, and then collecting and storing a culture supernatant and
(S2) isolating the extracellular vesicle from the culture supernatant.

13. The method of claim 10, wherein the culture supernatant is collected on the fourth day after transfection; the culture supernatant is stored at −80° C. for use; and the extracellular vesicle is isolated from the culture supernatant through ultracentrifugation.


SEQUENCE LISTING (5 sequences)

SEQ ID NO: 1 - DNA, 1536 bp, synthetic construct
  source 1..1536: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 2 - DNA, 1667 bp, synthetic construct
  source 1..1667: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 3 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 4 - DNA, 1184 bp, synthetic construct
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 5 - DNA, 500 bp, synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg